(19) United States
(12) Patent Application Publication
(10) Pub. No.: US 2020/0020043 A1
(43) Pub. Date: Jan. 16, 2020

(54) Condition-based Health Care Cost Estimation

(71) Applicant: ClaraPrice, Inc., Dallas, TX (US)

(72) Inventor: Sarah Soisson, Austin, TX (US)

(73) Assignee: ClaraPrice, Inc., Dallas, TX (US)

(21) Appl. No.: 16/509,436

(22) Filed: Jul. 11, 2019

Related U.S. Application Data
(60) Provisional application No. 62/696,753, filed on Jul. 11, 2018.

(51) Int. Cl. G06Q 40/08; G16H 10/60; G06F 9/54

(52) U.S. Cl. CPC G06Q 40/08; G06F 9/542; G16H 10/60

(57) ABSTRACT

Embodiments of systems and methods for condition-based health care cost estimation are described. In an embodiment, a method may include obtaining health care cost information associated with a Diagnosis-Related Group (DRG) defined by the tenth (10th) revision of the International Classification of Disease and related health problems (ICD-10) from a database. Such a method may further include receiving patient profile information from a remote user via a user portal interface. In an embodiment, the method may also include converting the patient profile information into a cost estimate based on the health care cost information using a processing device. The method may also include communicating the cost estimate to the remote user interface device.

### FIELD

This disclosure relates generally to health care cost estimation, and more specifically, to methods and systems for condition-based health care cost estimation.

### BACKGROUND

Healthcare pricing is a complex and rapidly changing area where there needs to be an automated and immediate response that is currently unable to be produced because diagnoses are continually evolving and procedures change in real time as surgery or other treatments are performed.

The International Classification of Diseases and Related Healthcare Problems (ICD) is maintained and promulgated by the World Health Organization (WHO) to establish a standard diagnostic tool for epidemiology, health management and clinical purposes. Revisions of ICD-9-CM mirror changes in the medical and health care field. The U.S. has been using ICD-9-CM since 1979, and it is not sufficient to serve the health care needs of the future. The content is no longer clinically accurate and has limited data about patients' medical conditions and hospital inpatient procedures, the number of available codes is limited, and the coding structure is too restrictive. The United States began using ICD-10 to code and classify mortality data from death certificates in January 1999. The conversion from ICD-9 to ICD-10 had an effect on coders and the mortality data system as a whole, including the revision of instruction manuals and development of new medical software to replace the manual coding process. To that end, the National Center for Health Statistics (NCHS) created special software to automate coding of medical information on the death certificate, according to WHO rules.

ICD-10-CM/PCS code sets enhance the quality of data for tracking public health conditions (complications, anatomical location), improved data for epidemiological research (severity of illness, comorbidities). ICD-10 is helpful for measuring outcomes and care provided to patients, making clinical decisions, identifying fraud and abuse and designing payment systems/processing claims.

ICD-9-CM codes are very different than ICD-10-CM/PCS code sets. There are nearly 19 times as many procedure codes in ICD-10-PCS than in ICD-9-CM. There are nearly 5 times as many diagnosis codes in ICD-10-CM than in ICD-9-CM. ICD-10 has alphanumeric categories instead of numeric ones. The order of some chapters has changed, some titles have been renamed, and conditions have been grouped differently.

Some automated tools have attempted to use databases of claims data to estimate pricing. Claims data is generated from a chargemaster which is a list detailing the official rate charged by a hospital for individual procedures, services, and goods. Such systems are insufficient because claims data does not factor patient condition into the formula when calculating a cost estimate. Current pricing tools based on claims data confuse patients with price ranges that do not reflect any complications that occurred or pre-existing conditional factors that may impact the actual bills. Further, current pricing tools do not use data associated with ICD-10 records obtained from a third-party database for the purpose of providing condition-based pricing.

### SUMMARY

Embodiments of systems and methods for condition-based health care cost estimation are described. In an embodiment, a method may include obtaining health care cost information associated with a Diagnosis-Related Group (DRG) defined by the tenth (10th) revision of the International Classification of Disease and related health problems (ICD-10) from a database. Such a method may further include receiving patient profile information from a remote user via a user portal interface. In an embodiment, the method may also include converting the patient profile information into a cost estimate based on the health care cost information using a processing device. The method may also include communicating the cost estimate to the remote user interface device.

In one embodiment, converting the patient profile information into a cost estimate further comprises assigning a weighting factor to the costs defined by the DRG, the weighting factor representative of possible comorbid conditions which may factor into the cost, and the possible comorbid conditions derived from the patient profile information.

In an embodiment, a system for condition-based health care cost estimation may include a network interface configured to: obtain health care cost information associated with a Diagnosis-Related Group (DRG) defined by the tenth (10th) revision of the International Classification of Disease and related health problems (ICD-10) from a database; and receive patient profile information from a remote user via a user portal interface. Such a system may also include a processing device configured to convert the patient profile information into a cost estimate based on health care cost information using a processing device, wherein the network interface is further configured to communicate the cost estimate to a remote user interface device.

Another embodiment of a method for condition-based health care cost estimation may include receiving patient profile information from a user over a web-based portal, the patient profile information comprising at least one of patient demographic data, patient location data, patient health condition data, and patient procedure data. The method may also include automatically generating a database request in response to the patient profile information, the database request comprising at least one command responsive to the received patient profile information, and configured to access data in at least one of a database of patient profile information, a database of user transaction information, and a database of aggregated open source datasets of ICD-10-based Diagnosis Related Group (DRG) data for performing DRG grouper logic and calculating a DRG value in response to the received patient profile information. In a further embodiment, the method may include receiving information from at least one of the databases in response to the database request. Such a method may also include calculating a DRG weight factor in response to the received patient profile information and in response to the data received from the at least one database. Additionally, the method may include determining a condition-based health care cost estimate in response to the DRG weight factor and the received patient profile information. Also, the method may include communicating the condition-based health care cost estimate to the user over the web-based portal.

Another embodiment of a system for condition-based health care cost estimation may include an application user interface configured to receive patient profile information from a user over a web-based portal, the patient profile information comprising at least one of patient demographic data, patient location data, patient health condition data, and patient procedure data. Also, the system may include an application database server configured to automatically generate a database request in response to the patient profile information, the database request comprising at least one command responsive to the received patient profile information, and configured to access data in at least one of a database of patient profile information, a database of user transaction information, and a database of aggregated open source datasets of ICD-10-based Diagnosis Related Group (DRG) data for calculating a DRG value in response to the received patient profile information. The system may also include an application DRG calculation and grouper engine coupled to the application user interface and the application database server, the application calculation engine configured to: receive information from at least one of the databases in response to the database request; calculate a DRG weight factor in response to the received patient profile information and in response to the data received from the at least one database. In an embodiment, such a system may also include an application server configured to: determine a condition-based health care cost estimate in response to the DRG weight factor and the received patient profile information; and communicate the condition-based health care cost estimate to the user over the web-based portal.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a schematic diagram illustrating one embodiment of a system for condition-based health care cost estimation.

FIG. 2 is a schematic diagram illustrating another embodiment of a system for condition-based health care cost estimation.

FIG. 3 is a logical stack diagram illustrating one embodiment of a software system for condition-based health care cost estimation.

FIG. 4 is a schematic diagram illustrating one embodiment of an apparatus for condition-based health care cost estimation.

FIG. 5 is a schematic flowchart diagram illustrating one embodiment of a method for condition-based health care cost estimation.

FIG. 6 is a logical diagram illustrating one embodiment of a process for condition-based health care cost estimation for a specified medical procedure.

FIG. 7 is a schematic block diagram of one embodiment of a system for condition-based health care cost estimation.

FIG. 8 is a schematic block diagram of one embodiment of a system for condition-based health care cost estimation.

FIG. 9 is a schematic flowchart diagram illustrating one embodiment of a method for condition-based health care cost estimation.

FIG. 10 is a screenshot diagram illustrating one embodiment of a user interface of an application for condition-based health care cost estimation.

FIG. 11 is a screenshot diagram illustrating one embodiment of a user interface of an application for condition-based health care cost estimation.

FIG. 12 is a screenshot diagram illustrating one embodiment of a user interface of an application for condition-based health care cost estimation.

FIG. 13 is a screenshot diagram illustrating one embodiment of a user interface of an application for condition-based health care cost estimation.

FIG. 14 is a screenshot diagram illustrating one embodiment of a user interface of an application for condition-based health care cost estimation.

FIG. 15 is a screenshot diagram illustrating one embodiment of a user interface of an application for condition-based health care cost estimation.

FIG. 16 is a screenshot diagram illustrating one embodiment of a user interface of an application for condition-based health care cost estimation.

FIG. 17 is a screenshot diagram illustrating one embodiment of a user interface of an application for condition-based health care cost estimation.

FIG. 18 is a screenshot diagram illustrating one embodiment of a user interface of an application for condition-based health care cost estimation.

FIG. 19 is a screenshot diagram illustrating one embodiment of a user interface of an application for condition-based health care cost estimation.

FIG. 20 is a screenshot diagram illustrating one embodiment of a user interface of an application for condition-based health care cost estimation.

FIG. 21 is a screenshot diagram illustrating one embodiment of a user interface of an application for condition-based health care cost estimation.

FIG. 22 is a screenshot diagram illustrating one embodiment of a user interface of an application for condition-based health care cost estimation.

FIG. 23 is a screenshot diagram illustrating one embodiment of a user interface of an application for condition-based health care cost estimation.

FIG. 24 is a screenshot diagram illustrating one embodiment of a user interface of an application for condition-based health care cost estimation.

FIG. 25 is a screenshot diagram illustrating one embodiment of a user interface of an application for condition-based health care cost estimation.

FIG. 26 is a screenshot diagram illustrating one embodiment of a user interface of an application for condition-based health care cost estimation.

### DETAILED DESCRIPTION

Various features and advantageous details are explained more fully with reference to the nonlimiting embodiments that are illustrated in the accompanying drawings and detailed in the following description. Descriptions of well-known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those skilled in the art from this disclosure.

The present embodiments include systems and methods of health care cost estimation and bill payment. The parties to the process, which typically include the patient and the provider, select from a number of choices. This selection of information includes the bill, payments, patient information, medical history, patient conditions, and further actions of the patient and providers. An accumulation of choices by the involved parties can include digital information in each step. The digital data is stored electronically in a data storage system, and used to review, calculate, analyze, and negotiate prices at multiple different settings.

Assigning a price to diagnosis and treatment cannot be done in a manual way, due to the complex nature of Diagnosis Related Groups (DRGs) decision tree processes. There are many advantages of the present embodiments, as DRGs are now used and proven worldwide for accurate, universal healthcare pricing and reimbursement. DRGs are weighted differently due to conditional factors assigned to individual cases.

Most other healthcare pricing tools generate one price range excluding individual conditional factors. Consumers are overwhelmed with hospital bills, frustrated with vague price ranges and limitations to providers they can choose from, and reluctant to negotiate with Hospital financial teams bogged down with fee-based payment models. The present embodiments accelerate the adoption of alternative pricing models in hospitals with complex billing processes and outdated chargemasters.

Accurate hospital bill estimates empower consumers when choosing hospital care locations, understanding the cost of their individual case and lowering bills after treatment. Other pricing tools give range of prices, can limit the providers available, and include bloated claims data. Hospital Financial counselors can offer the estimates in negotiations with patient billing inquiries. Current billing processes rely on chargemasters, which are prone to errors, and get bogged down with fee-based payment models and overwhelm revenue cycle teams. Revenue cycle teams trust ICD-10 data and can use DRG-based pricing to associate with and communicate prices to patients.

Self-funded employers can add access to the estimating tool in their on-demand healthcare benefits packages, and get aggregated data on their employees' billing claims to negotiate reduced costs with insurance companies. Employers with self-funded insurance or Reference Based Pricing (RBP) models can reduce costs with contractual Healthcare provider relationships. In an embodiment, the employer's data may compare to a Hospital's claims data and Case Mix Index (CMI).

The present embodiments are based on DRG logic and grouping standards and focused on actual pricing formulas from accepted data sources, such as ICD-10. Beneficially, the present embodiments provide greater clarity and accuracy for individuals seeking estimates of health care costs prior to treatment. In some embodiments, costs are available prior to treatment, during treatment and the final and most accurate cost estimate should be calculated after discharge. An example is a patient with a post-op infection which impacts the length of stay and the resource intensity of services performed. The pre-op cost could not reflect an unknown event such as post-op infections or a heart attack. The present embodiments do not deliver cost estimates with a range of prices based on claims data. Rather, the cost estimates are precisely derived from each patient's condition and location. Other estimating tools fuel the Fee-for-service (FFS) model that creates itemized bills from chargemasters which are prone to error and typically contain flawed data. Additionally, the present embodiments drive value-based payment models. For example, the described embodiments may provide a functioning payment model that eliminates FFS generated bills. When a hospital submits to get paid, cost and quality may be intertwined. The DRG system enables the pay-for-value model, as it specifies detailed diagnoses. Furthermore, the present embodiments provide an affordable healthcare catalyst. The described embodiments establish relationships between patients and providers to reach cost affordability for healthcare services delivered. Patients gain the power to prepare financially, understand their bill, and shop for care. Hospitals welcome users of a collective cost estimate solution, and benefit from up-front commitments to pay by improving the patient experience.

In an embodiment, the process for condition-based health care cost estimation includes multiple functional steps, which are not necessarily described in any particular order herein. For example, the process may include the following steps:

STEP 1: Patient logs in to patient portal through their company's custom HR portal. The patient portal may be considered white label software and will appear seamlessly in benefit packages and hospital financial counselor's desktop tools.

STEP 2: Patient completes online case profile capturing their condition and prognosis. Here's an example of questions that would be asked:
Does the patient expect to stay overnight in the Hospital? If yes, please continue.
Does the patient have any pre-existing conditions? Diabetes, Hypertension, Emphysema, if yes please continue.
Did the patient experience any complications during surgery if one was performed? A post-op infection or rupture of the appendix?

STEP 3: A processing device analyzes the conditional factors in step 2 to group the Patient into a DRG and provide a cost estimate. Educating the patient and provider regarding age and personal health conditions enables some degree of price transparency.

STEP 4: Patient enters demographic factors to indicate biological sex, location, and zip code of where they wish to be treated. This price quote is “shoppable”.

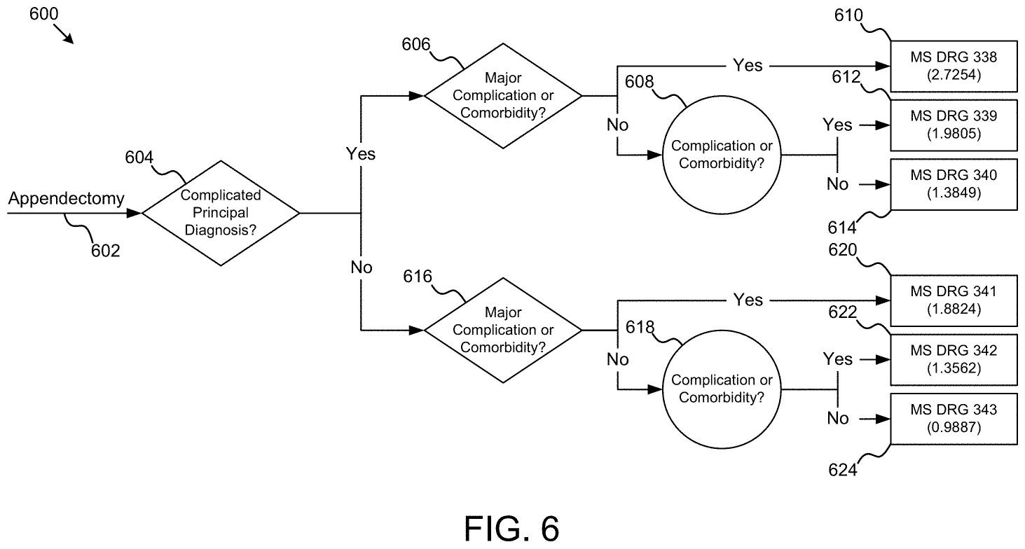

STEP 5: The processor processes these factors in conjunction with corresponding DRG data, performing DRG grouping logic, assigning the Patient to a DRG, and providing a cost estimate for the patient's individual case. The estimated cost in the embodiment described in FIG. 6 is $16,400. (Based on ICD-10 data and DRG weight, though the DRG weight may change and will be updated regularly to reflect changes in CMS data)

STEP 6: Patient is armed with an accurate healthcare price estimate to negotiate the cost of care before, during and after treatment.

Thus, according to the described embodiments, simply by logging in to a secure portal and answering a set of questions about a patient's prognosis, condition, situation and location, patients and providers can receive a cost estimate for their particular health episode at an acute care hospital before, during and after care. In such an embodiment, the estimate may clearly state and explain the calculated DRG and price, based on ICD-10 data approved worldwide and in the U.S. for Medicare and Medicaid cases. Patient data is secure and not identifiable by patient or PHI. In an embodiment, all identifying data may be converted to an arbitrary patient ID to control the numbers and retain the clinical data only. Data can be shared in aggregate form also known as a Case Mix Index (CMI) to evaluate and plan for the severity of illness and resource utilization of a population (for an employer to evaluate cost of self-insuring employees).

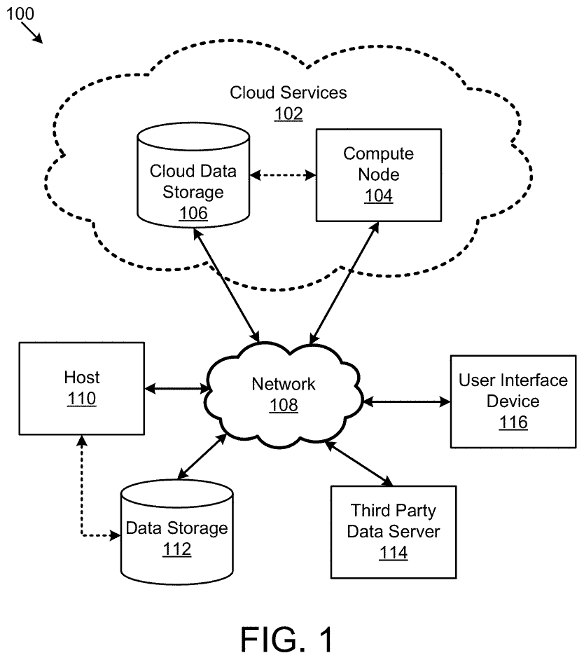

The present embodiments include systems, methods, and apparatuses for condition-based health care cost estimation. FIG. 1 illustrates one embodiment of a system 100 for condition-based health care cost estimation. In an embodiment, the system 100 may include cloud services 102 may be specially configured for hosting applications and data described in accordance with the present embodiments. Specifically, the cloud services 102 may include one or more compute nodes 104 configured to run operations, process data, and conduct communications associated with the described methods and processes. In addition, cloud services 102 may include cloud data storage 106 for storing one or more sets of data described herein. In a specific embodiment, the cloud data storage 106 may be extensible, thereby allowing dynamic expansion of the system as the volume of user data and available third-party data grows.

In an alternative or additional embodiment, certain components of the system 100 may be operated on a stand-alone or independent host 110 and associated data storage device 112. The cloud services 102 and/or host 110 may communicate with a third-party data server 114 via network 108 to obtain health care cost information associated a Diagnosis-Related Group (DRG) defined by a by the tenth (10th) revision of the International Classification of Disease and related health problems (ICD-10). Additionally, the cloud services 102 and/or 110 may communicate with user interface device 116 over network 108. The user interface device 116 may display a patent portal interface for receiving patient profile information from a remote user. The host 110 and/or the compute node(s) 104 may convert the pateint profile information into a cost estimate based on health care cost information and communicate the cost estimate to a remote user interface device 116 via network 108.

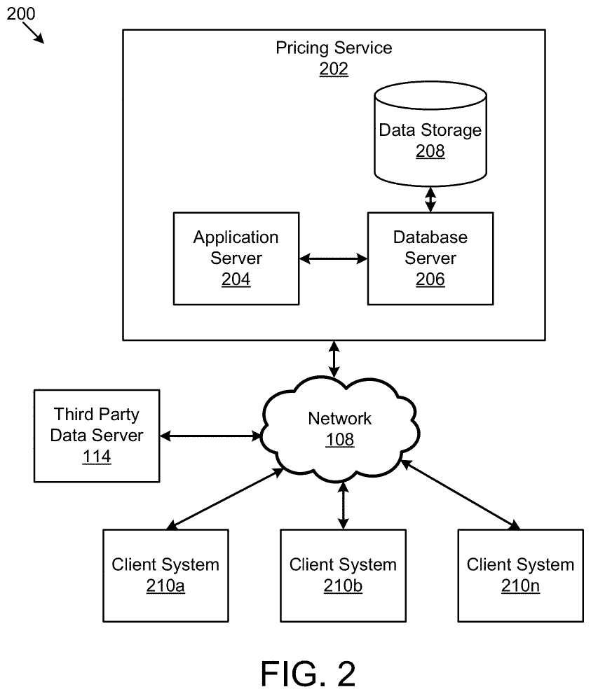

FIG. 2 illustrates a further embodiment of a system 200 for condition-based health care cost estimation. In such an embodiment, the system 200 includes a pricing services system 202 comprising an application server 204 a database server 206 and a data storage device 208. In one embodiment, the system 200 may be implemented on cloud services 102. In an alternative embodiment, the pricing services 202 may be implemented on the host 110 and data storage device 112. The pricing services 202 may communicate via network 108 with a third-party data server 114 and one or more client systems 210a-n. In one embodiment, the client systems 210a-n may be implemented on user interface device 116. In an embodiment, the client systems 210a-n may be specially configured to operate a client application, comprising a patient portal interface, which is configured to communicate via network 108 to the pricing services 202. Thus, in such an embodiment, client systems may operate a lightweight client application that operates the patient portal interface. Beneficially, such an embodiment may maintain the bulk of data, including third-party pricing data and patient data for other users on the pricing services. For example, a first user may provide patient profile information via client system 210a and a second user may provide patent profile information via client system 210b. In such an embodiment, each of client system 210a-b may be communicated separately to pricing services 202 via network 108 and stored in a separate data storage virtual container in data storage 208. Thus, user confidentiality may be maintained between the first user and the second user.

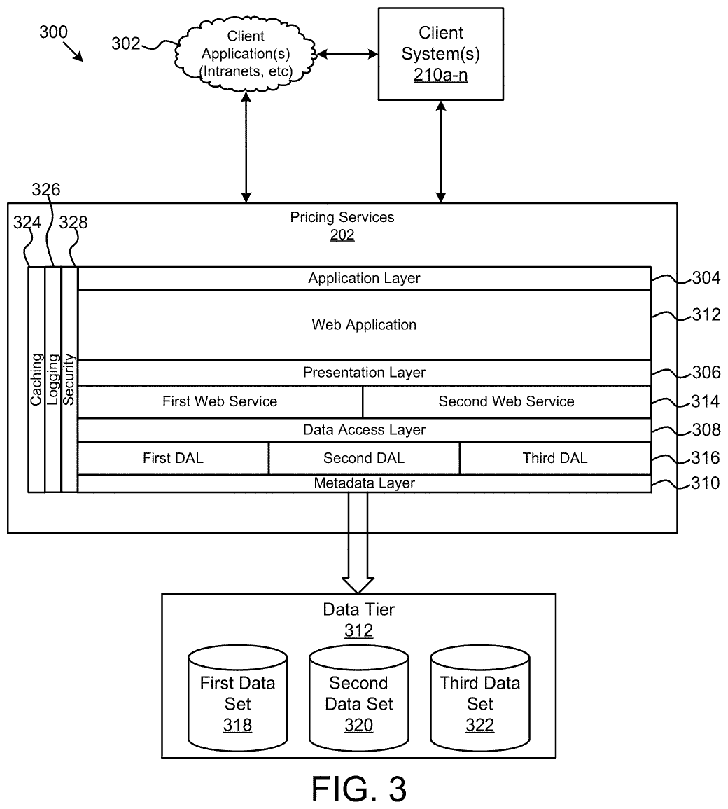

FIG. 3 illustrates one embodiment of a network-based system 300 for condition-based health care cost estimation. In one embodiment, the network-based system 300 includes pricing services 202. Additionally, the network-based system 300 may include one or more client systems 210a-n. In still a further embodiment, the network-based system 300 may include one or more network-based client applications 302 configured to be operated over a network 108 including an intranet, the Internet, or the like. In still another embodiment, the network-based system 300 may include one or more data storage devices 104.

The network-based system 300 may include components or devices configured to operate in various network layers. For example, the pricing services 202 may include modules configured to work within an application layer 304, a presentation layer 306, a data access layer 308 and a metadata layer 310. In a further embodiment, the pricing services 202 may access one or more data sets 318-322 that comprise a data layer or data tier 312. For example, a first data set 318, a second data set 320 and a third data set 322 may comprise a data tier 312 that is stored on one or more data storage devices 112, in one embodiment.

One or more web applications 312 may operate in the application layer 304. For example, a user may interact with the web application 312 though one or more I/O interfaces 318, 320 configured to interface with the web application 312 through an I/O adapter 310 that operates on the application layer. In one particular embodiment, a web application 312 may be provided for condition-based health care cost estimation that includes software modules configured to perform the steps of the condition-based health care pricing estimation process described herein.

In a further embodiment, the server 102 may include components, devices, hardware modules, or software modules configured to operate in the presentation layer 306 to support one or more web services 314. For example, a web application 312 may access or provide access to a web service 314 to perform one or more web-based functions for the web application 312. In one embodiment, a web application 312 may operate on a first server 102 and access one or more web services 314 hosted on a second server (not shown) during operation.

For example, a web application 312 for displaying the patient portal interface, or other information may access a first web service 314 for providing a list of health care providers in a selected geographical area and a second web service 314 for displaying a price comparison of health care service pricing. The web services 314 may receive the patient profile data. In response, the web service 314 may return data pricing data. One of ordinary skill in the art will recognize various web-based architectures employing web services 314 for modular operation of a web application 312.

In one embodiment, a web application 312 or a web service 314 may access one or more of the data sets 318-322 through the data access layer 308. In certain embodiments, the data access layer 308 may be divided into one or more independent data access layers 316 for accessing individual data sets 318-322 in the data tier 312. These individual data access layers 316 may be referred to as data sockets or adapters. The data access layers 316 may utilize metadata from the metadata layer 310 to provide the web application 312 or the web service 314 with specific access to the data set 312.

For example, the data access layer 316 may include operations for performing a query of the data sets 318-322 to retrieve specific information for the web application 312 or the web service 314. In a more specific example, the data access layer 316 may include a query for third party pricing data from a third-party data server 114.

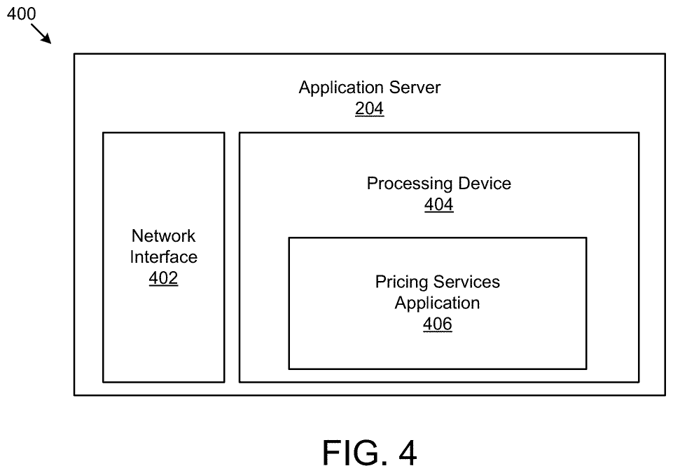

FIG. 4 illustrates an embodiment of an apparatus 400 for condition-based health care pricing estimation. In an embodiment, the apparatus 400 includes the application server 204, which may be specially configured and operated on either the host 110 or the compute nodes 104. In such an embodiment, the application server 204 may include a network interface 402 configured to obtain health care cost information associated a Diagnosis-Related Group (DRG) defined by a by the tenth (10th) revision of the International Classification of Disease and related health problems (ICD-10) from a third-party database, and to receive patient profile information from a remote user via a patient portal interface. Additionally, the application server 204 may include a processing device 404 specially configured to perform operations defined by a pricing services application 406. In an embodiment, the processing device 404 may be configured to convert the patient profile information into a cost estimate based on health care cost information using a processing device. In such an embodiment, the network interface 402 may be further configured to communicate the cost estimate to a remote user interface device.

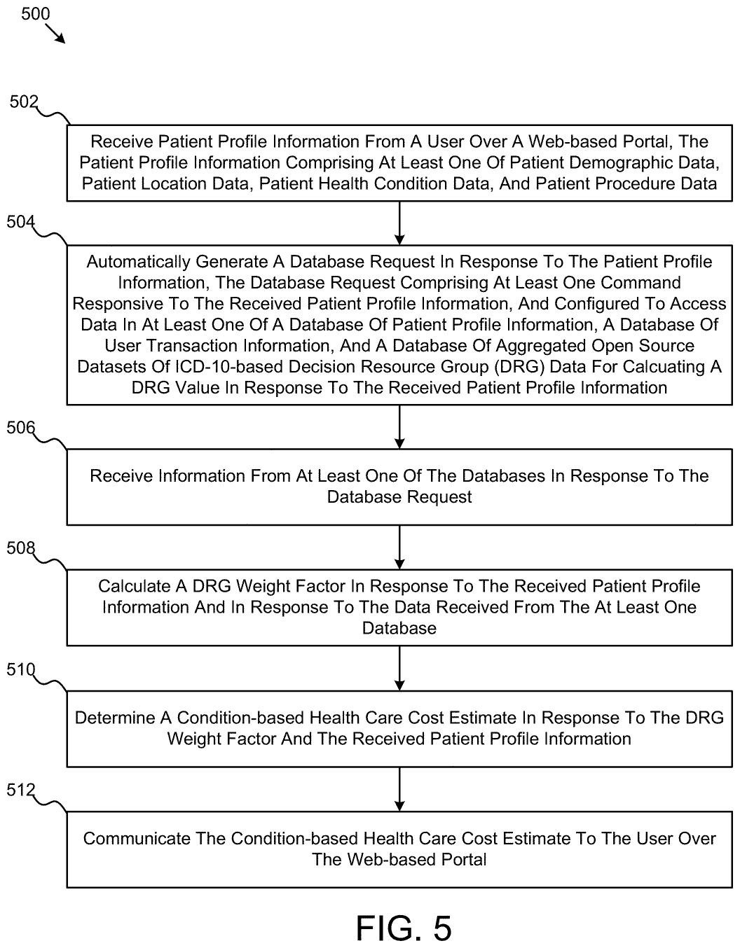

FIG. 5 illustrates one embodiment of a method 500 for condition-based health care pricing estimation. In one embodiment, the method 500 may include receiving patient profile information from a user over a web-based portal, the patient profile information comprising at least one of patient demographic data, patient location data, patient health condition data, and patient procedure data, as shown at block 502. At block 504, the method 500 includes automatically generating a database request in response to the patient profile information, the database request comprising at least one command responsive to the received patient profile information, and configured to access data in at least one of a database of patient profile information, a database of user transaction information, and a database of aggregated open source datasets of ICD-10-based Decision Resource Group (DRG) data for calculating a DRG value in response to the received patient profile information. The method 500 also includes receiving information from at least one of the databases in response to the database request, as shown at block 506. At block 508, the method 500 includes calculating a DRG weight factor in response to the received patient profile information and in response to the data received from the at least one database. The method 500 also includes determining a condition-based health care cost estimate in response to the DRG weight factor and the received patient profile information as shown at block 510 and communicating the condition-based health care cost estimate to the user over the web-based portal as shown at block 512.

FIG. 6 illustrates one example of how the methods and systems described herein may be implemented to generate a precise health care cost estimate for a specific medical procedure. According to the embodiment of FIG. 6, the patient indicates, via the patient portal interface, that an appendectomy procedure is required. First, it is determined whether the principal diagnosis of appendicitis is complicated, for example by rupture of the appendix. Regardless of the answer, a second determination of whether a major complication or comorbidity is indicated in the patient profile. If the principal diagnosis is complicated and a major complication or comorbidity is present, then MSDRG 338 with a weight factor of 2.7254 is indicated. If no major complication or comorbidity is indicated, but a minor complication or comorbidity is indicated then DRG 339 with a weighting factor of 1.9805 is indicated. If no complication is present, then a DRG 340 with a weighting factor of 1.3849 is indicated. In the case of no complication of principal diagnosis is present, then DRGs 341, 342, and 343 may be indicated with weighting factors 1.8824, 1.3562, and 0.9887 respectively.

In such embodiments, the DRG may include pricing information derived from ICD-10 information. In an embodiment, a local cost estimate is multiplied by the weighting factor associated with the DRG to calculate a cost estimate for the procedure. Thus, the processing device 404 may convert the patient profile information into a cost estimate based on health care cost information using a processing device.

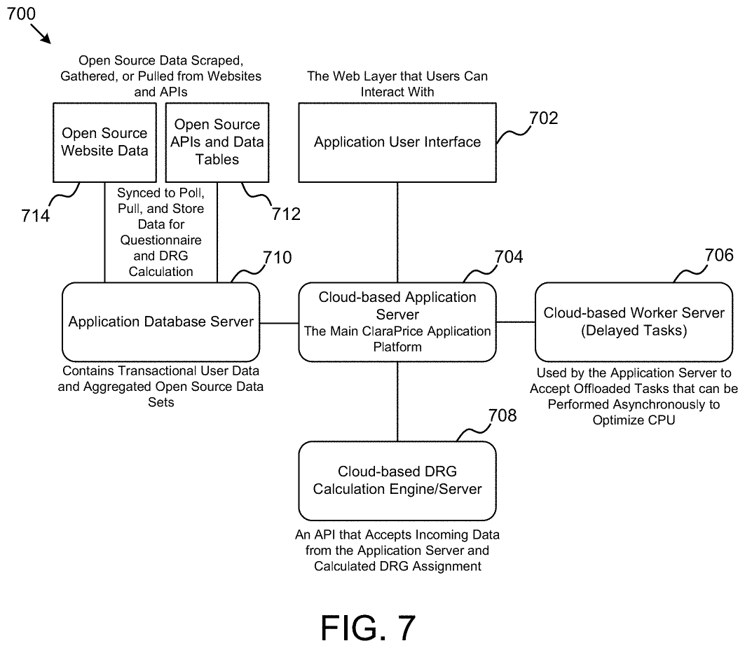

FIG. 7 illustrates another embodiment of a system for condition-based health care cost estimation. In an embodiment, the system includes an application user interface. In an embodiment, the application user interface may include a web layer that users can interact with. The system may also include a cloud-based application server. The application server may be the central engine for handling condition-based health care cost estimation functions and operations. The system may further include an application database server configured to automatically scrape data from one or more open source websites and one or more open source APIs and data tables. The application database server may sync to poll, pull and store data for questionnaire and DRG calculations. Additionally, the application database server may include transactional user data and aggregated open source data sets. The system may also include a cloud-based DRG calculation engine or server. In an embodiment, this component may provide an API that accepts incoming data from the application server and calculates a DRG assignment. In some embodiments, the system may include a cloud-based worker server configured to accept offloaded tasks from the application server that can be performed asynchronously to optimize system performance. One of ordinary skill may recognize alternative or additional system implementations.

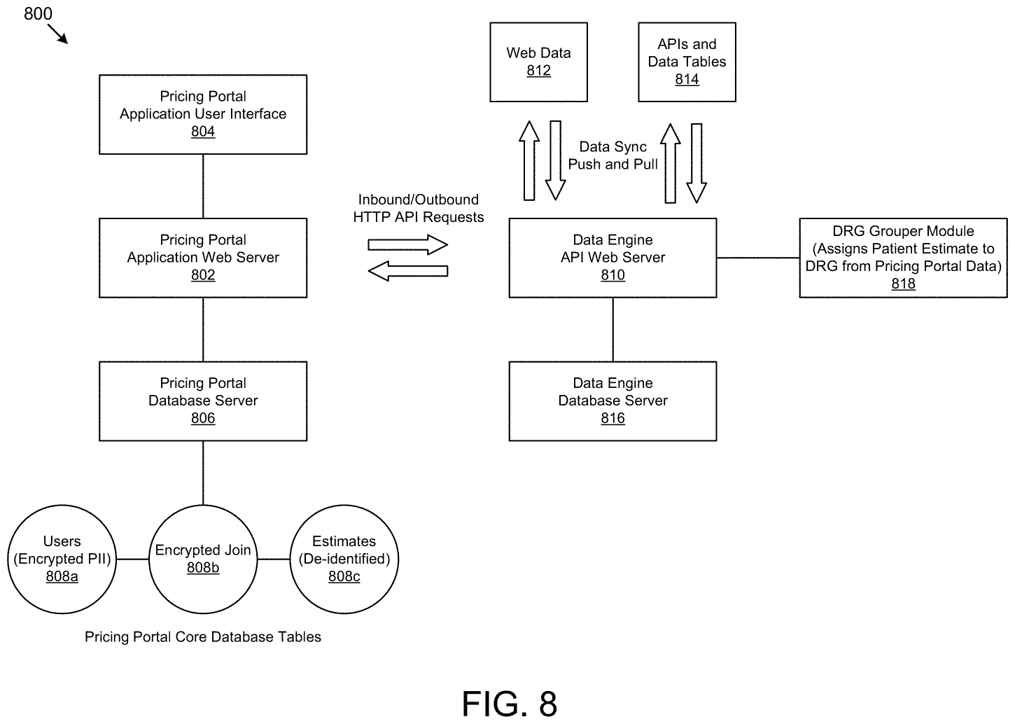

FIG. 8 illustrates another embodiment of a system 800 for condition-based health care cost estimation. In an embodiment, the system 800 includes an application web server 802 and a pricing portal user interface device 804. The application web server 802 may be coupled to a database server 806 which interfaces a plurality of databases 808a-808c. In one embodiment, the databases 808a-808c may include a user data database 808a, an encrypted user credential database 808b, and a de-identified estimates database 808c. The system 800 may further include a data engine 810 configured to perform calculations in response to information entered by the user at the user interface 804 and in response to web data 812 and online data tables 814. Additionally, the data engine may connect to a data engine database server 816.

In an embodiment, system 800 may further include a DRG grouper module 818 configured to assign a patient estimate to a DRG from the pricing portal data. In such an embodiment, the DRG grouper module 818 may be configured to convert the patient profile data into ICD-10 data for DRG grouping, post the converted data set over HTTP API request to perform the DRG grouping, and return the assigned DRG to the patient pricing portal over HTTP API request. In such an embodiment, the grouped DRG data and patient profile may be used to determine the cost estimate.

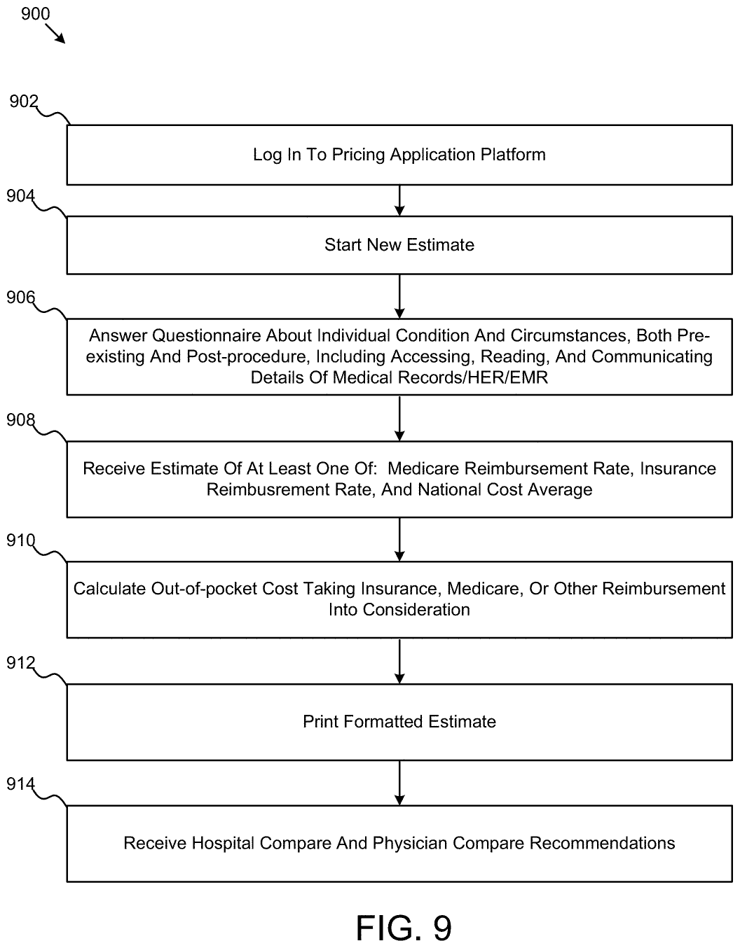

FIG. 9 illustrates one embodiment of a method 900 of using the system 800 of FIG. 8. In an embodiment, the method 900 includes logging into the pricing application platform at block 902. At block 904, the method includes starting a new estimate. At block 906, the method 900 includes answering a questionnaire about individual condition and circumstances, both pre-existing and post-procedure, including assessing, reading, and communicating details of medical records, EHR and EMR data. At block 908, the method 900 includes receiving an estimate of at least one of: medical reimbursement rates, insurance reimbursement rates, and national cost averages. At block 910, the method includes calculating out-of-pocket costs for the procedure, taking insurance, Medicare or other reimbursement into consideration. At block 912, the method 900 includes printing a formatted estimate. At block 914, the method 900 includes receiving facility comparison and/or provider comparison information.

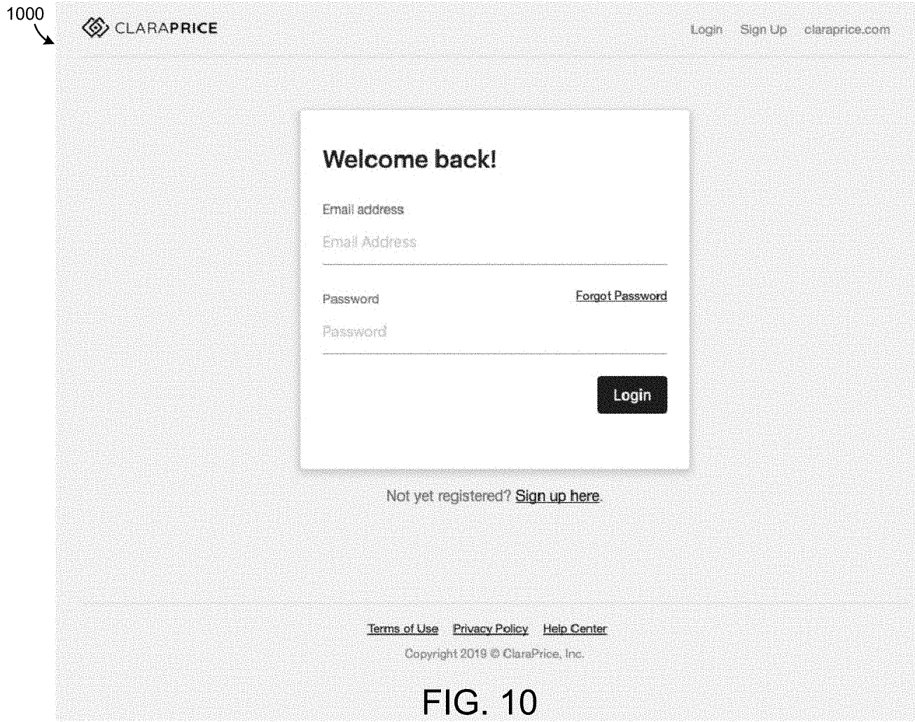

The screenshots shown in FIGS. 10-26 illustrate an example application flow which may be produced in response to certain selected demographic information, procedure information, diagnosis information, and pre-existing condition information. The result of the application flow is a formatted cost estimate, as well as facility and provider comparison data. In an embodiment, the screens 1000-2600 may be displayed on an embodiment of a pricing portal user interface device.

FIG. 10 is a screenshot diagram of one embodiment of a user interface of an application for condition-based health care cost estimation. At screen 1000, a user may enter login information. For example, the user may enter a username, an email address, or some other unique identifier. Additionally, the user may enter a password for securely accessing the user's account. Screen 1000 may also include links to pages to allow a user to set up a new user account or retrieve forgotten user account information, such as forgotten usernames or passwords.

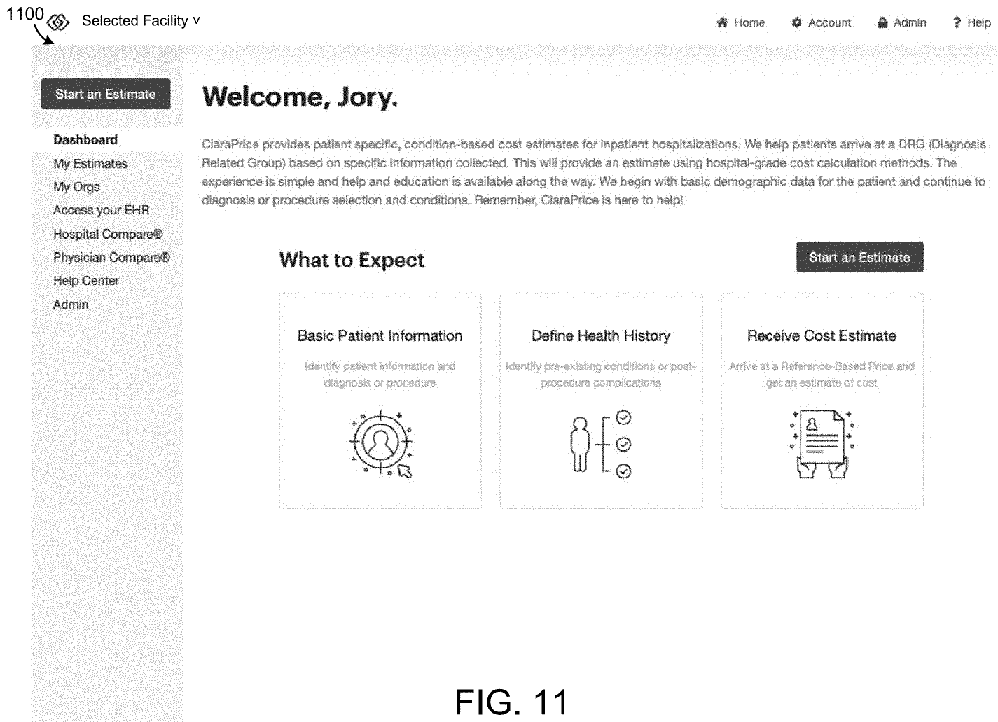

FIG. 11 is a screenshot diagram of a user interface showing a user dashboard view. At screen 1100, the user interface may include a landing page with a welcome message to a user, instructions for the user, one or more links or navigation bars, or other controls for initiating the health care pricing application. For example, at screen 1100 the user is welcomed with a welcome message and a navigation menu is provided. Additionally, the user is given information or instructions on what to expect during operation of the application. A button or other user control allows the user to initiate a pricing estimate.

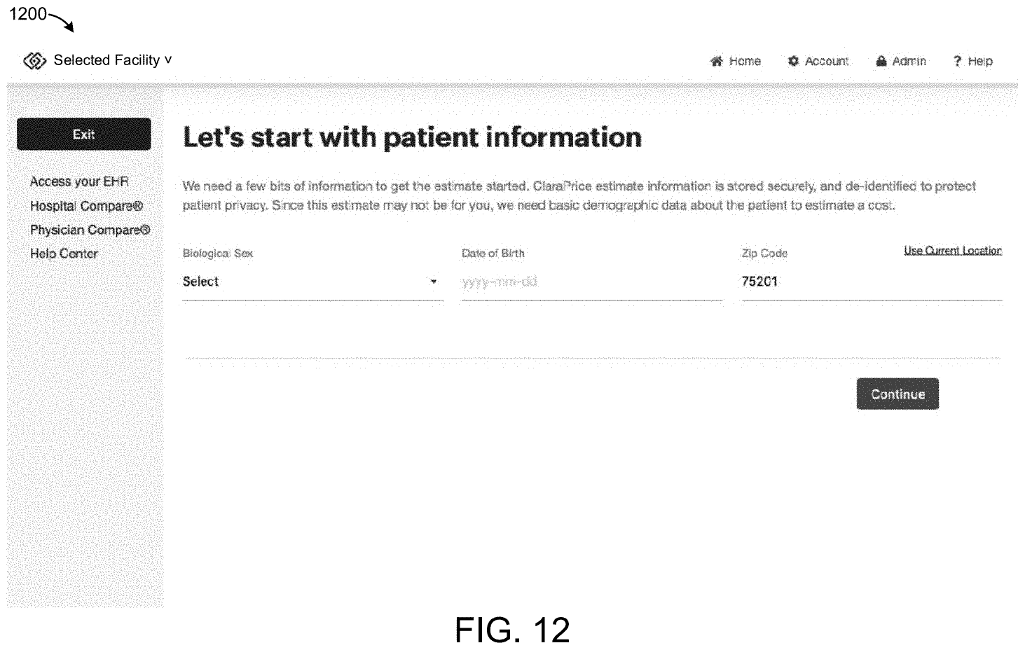

FIG. 12 is a screenshot diagram of a user interface showing a patient information questionnaire. At screen 1200, the user is prompted to provide demographic information including, but not limited to, gender identification, date of birth or age identification, zip code or city/state identification, and the like. A further user control, such as a button labeled “continue” is provided to allow the user to navigate to a next screen. Additionally, further controls allowing the user to exit the program or access additional information is provided in a navigation bar.

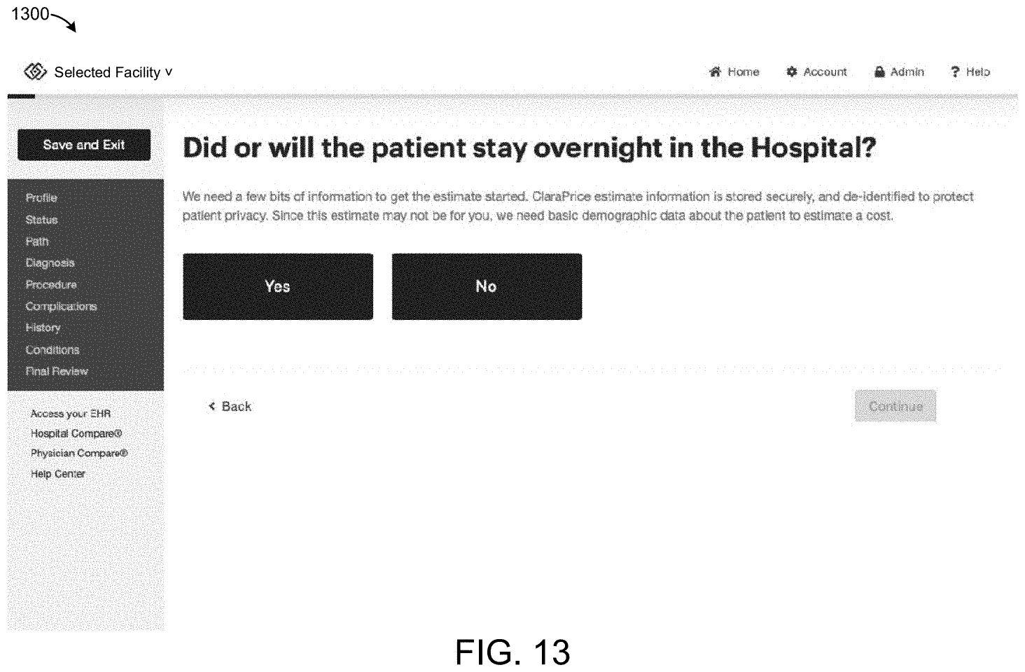

FIG. 13 is a screenshot diagram of a user interface showing another patient information questionnaire page. At screen 1300 the user is asked whether a patient has or will stay overnight in a hospital. User controls are provided to allow the user to provide responsive inputs. Further user controls are provided to navigate the questionnaire. Additionally, one or more links may be included in a navigation bar for allowing the user to navigate the questionnaire pages and other informational pages.

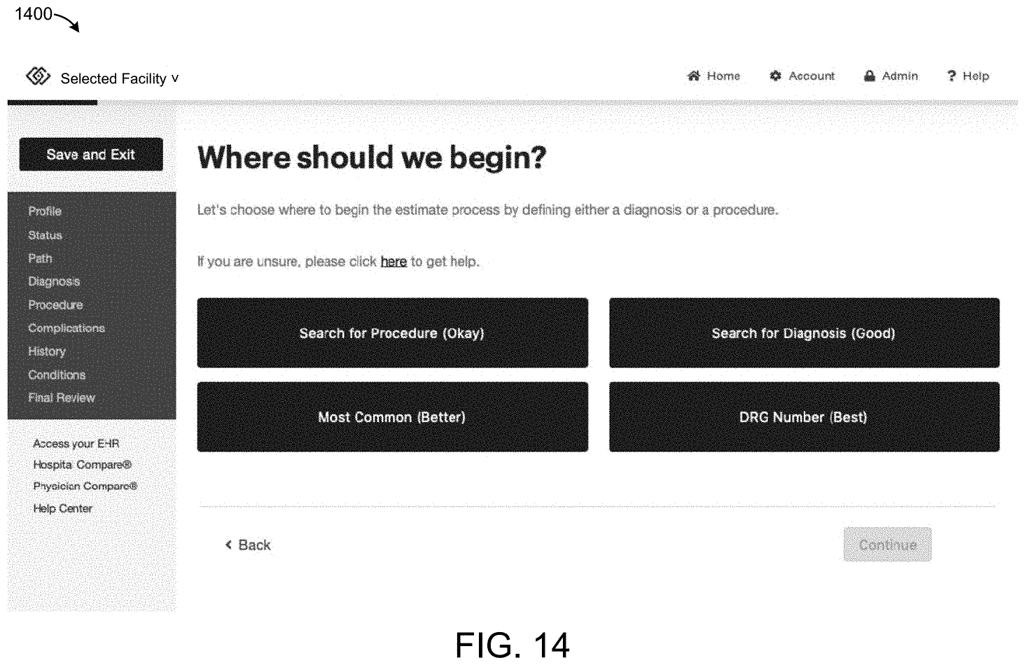

FIG. 14 is a screenshot diagram of a user interface showing another patient information questionnaire page. At screen 1400, the user is given options for identifying either a diagnosis or a procedure. In the embodiment of screen 1400, the user is provided with multiple user controls. In a further embodiment, screen 1400 includes a first button for allowing the user to search for a procedure in a predetermined list of procedures. A second button allows the user to search for a diagnosis from a predetermined list of diagnoses. A third button allows a user to search a list of most common procedures or diagnoses. In one embodiment, the most common list may be predetermined. In another embodiment, the most common list may be adaptive to the patient's demographic data previously entered. A fourth button allows a user to enter a DRG number corresponding to a procedure or diagnosis directly. Further user controls allow the user to navigate the questionnaire.

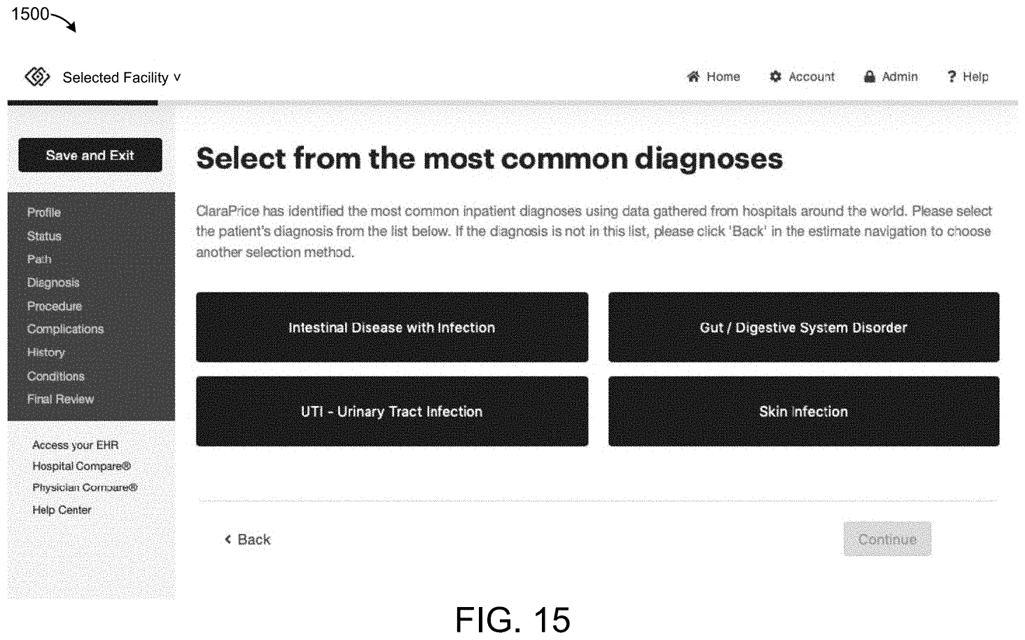

FIG. 15 is a screenshot diagram of a user interface showing another patient information questionnaire page. In one embodiment, screen 1500 may be presented by the application web server to the application user interface device in response to a selection of the third button for a search of a list of most common procedures or diagnoses at screen 1400. In one such embodiment, further selectable objects associated with one or more common diagnoses or procedures may be presented. For example, in the embodiment of FIG. 15, a first selectable object is presented for a diagnosis of “intestinal disease with infection,” a second selectable object is presented for a diagnosis of “gut/digestive system disorder,” a third selectable object is presented for “UTI—Urinary Tract Infection,” and a fourth selectable object is presented for “skin infection.” A user may select one or more selectable objects and then navigate to a next screen. In such an embodiment, the user's selection may be transmitted from the application user interface to the application web server. Similar screens (not shown) may be presented for other user selections at screen 1400.

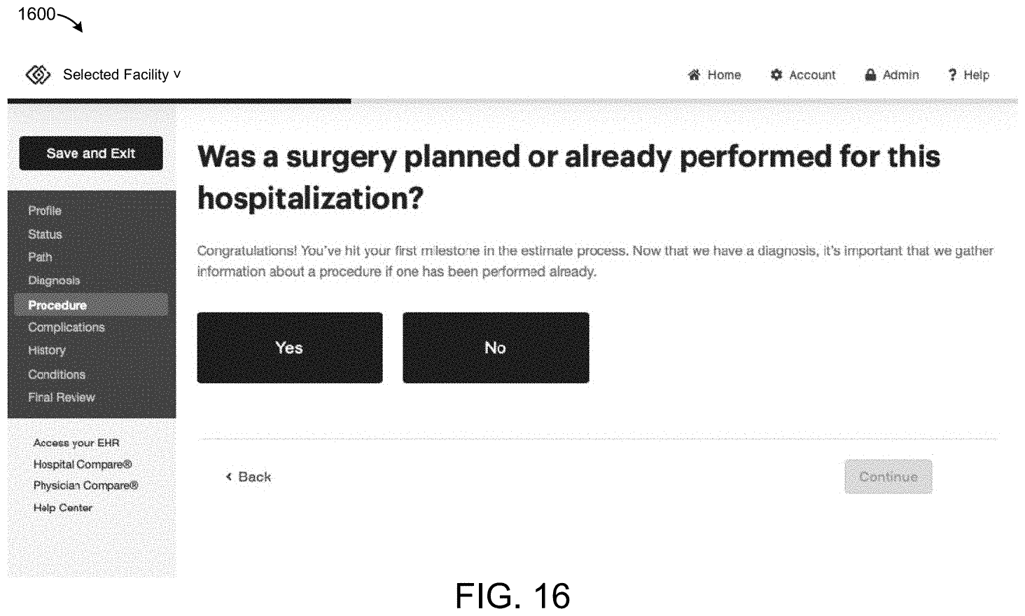

FIG. 16 is a screenshot diagram of a user interface showing another patient information questionnaire page. In one embodiment, screen 1600 may include prompts for allowing a user to select whether a surgery is planned or has already been performed, either in association with a hospital visit or on an outpatient basis. The user may be presented with one or more selectable objects, such as buttons to provide user input. In addition, as shown at FIG. 16, the user may be provided with encouragement to help him/her complete the questionnaire.

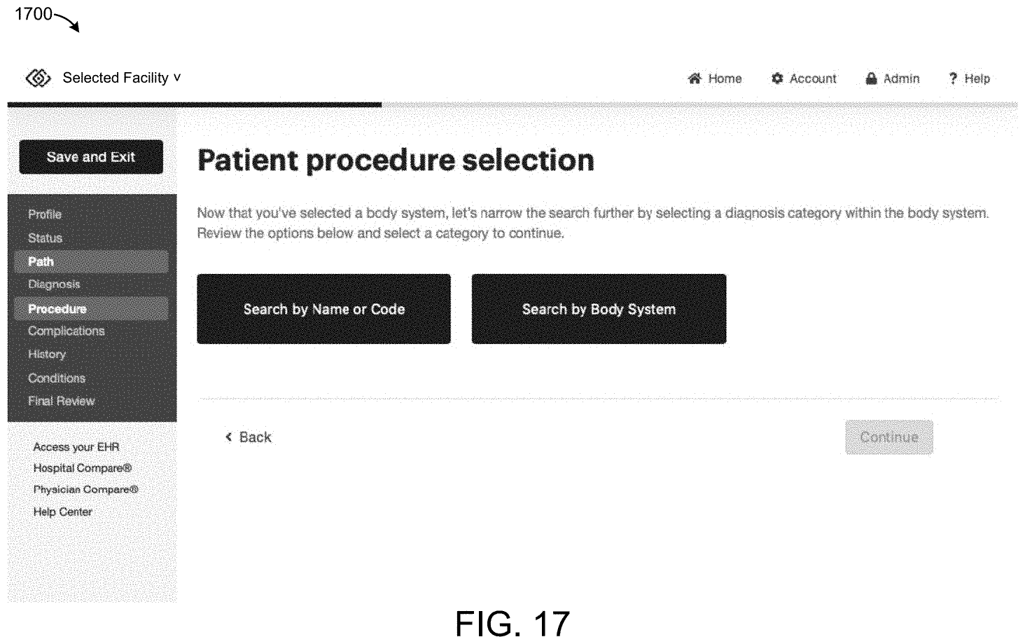

FIG. 17 screenshot diagram of a user interface showing another patient information questionnaire page. In an embodiment, at screen 1700 the user may be provided with a selectable list of options to select a procedure to be performed. For example, as illustrated in FIG. 17, a user may be provided with one or more selectable objects, such as the selectable object labeled “search by name or code” and selectable object labeled “search by body system.”

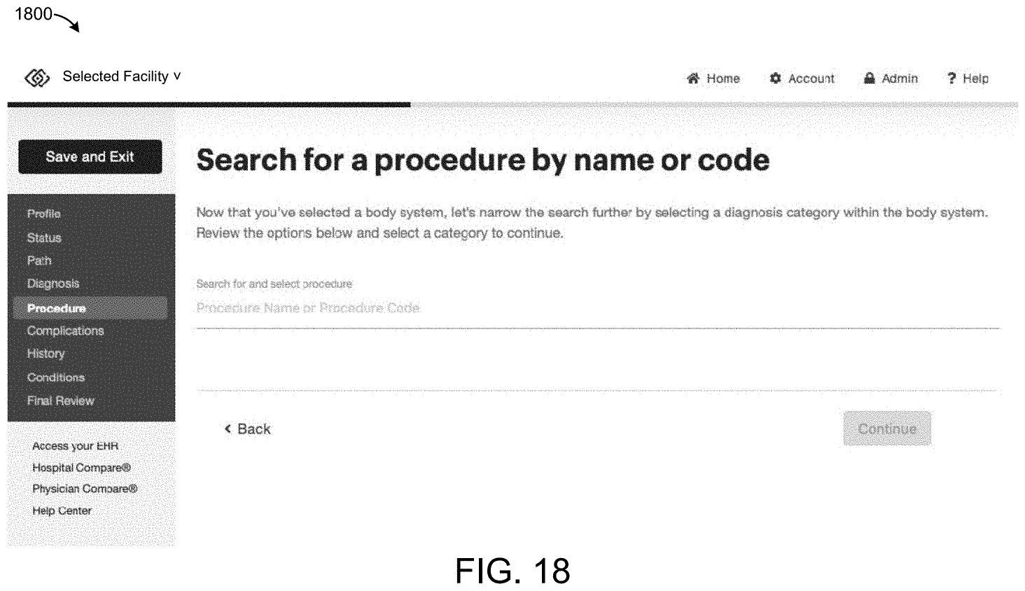

If at screen 1700 the user selects “search by name or code,” then screen 1800 as shown at FIG. 18 may be displayed. Screen 1800 may include a search field, and inputs entered by the user may be transmitted to the application web server, which may then generate a query for the database server to search for a selected procedure name or procedure code in one or more of the databases shown in the systems of FIGS. 1-3 or FIGS. 7-8.

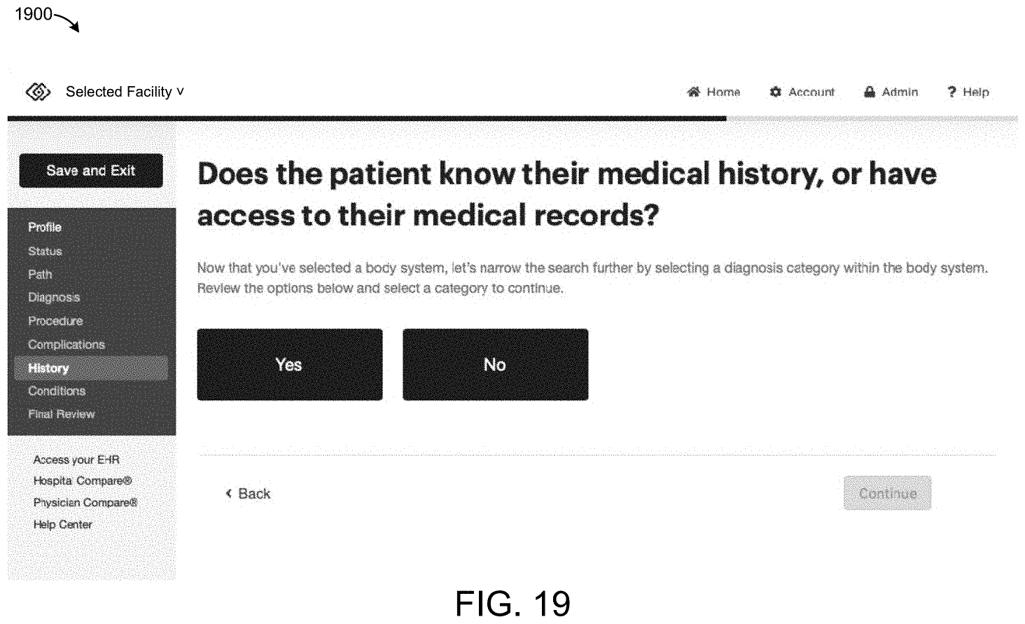

Once a procedure has been identified, a screen 1900 may be displayed to prompt the user for medical history or medical records. Selectable objects may be displayed to allow the user to select whether medical history or medical records are available. FIG. 19 illustrates one embodiment of the screen 1900 for determining whether medical history or medical records are available.

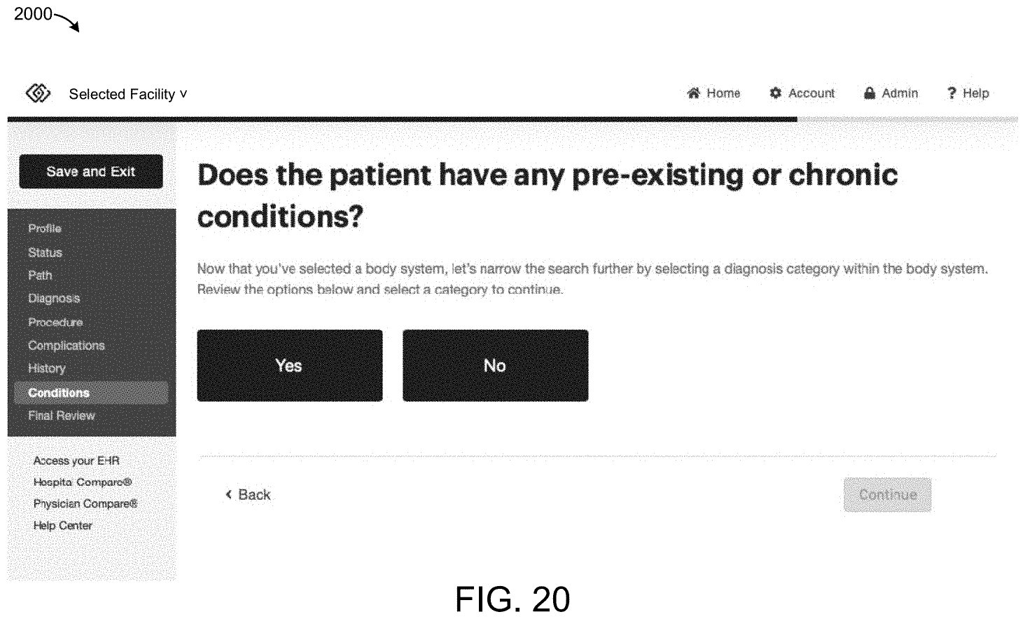

FIG. 20 is a screenshot diagram of a user interface showing another patient information questionnaire page. In screen 2000 shown in FIG. 20, the user is prompted to select whether or not the patient has any pre-existing or chronic medical conditions. One or more selectable objects may be displayed to allow the user to make the selection.

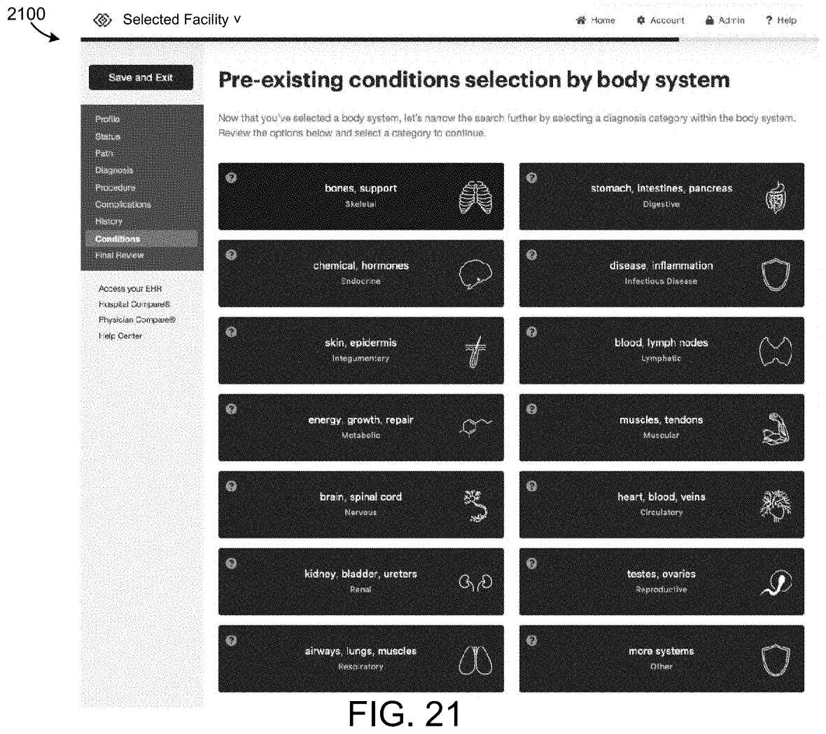

FIG. 21 is a screenshot diagram of a user interface showing another patient information questionnaire page. FIG. 21 illustrates screen 2100, which is an embodiment of a user interface for allowing a user to identify one or more pre-existing conditions or chronic illnesses for the patient. In an embodiment, screen 2100 includes one or more selectable objects representing various body systems. One or more body systems may be elected by the user in response to clicking on one or more of the selectable objects. Although the embodiment of FIG. 21 specifically illustrates a method of selection pre-existing conditions by body system, one of ordinary skill will recognize that alternative methods may be used, including accessing patient records in on-line or remotely managed databases, allowing the user to input natural language, allowing the user to input codes associated with the pre-existing condition, or the like.

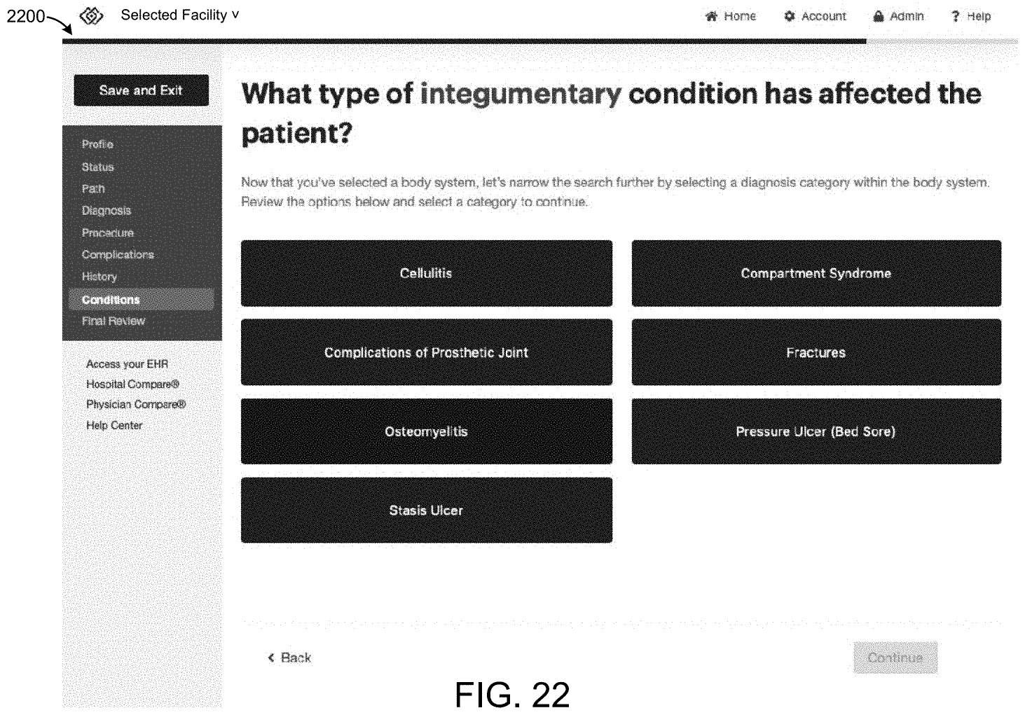

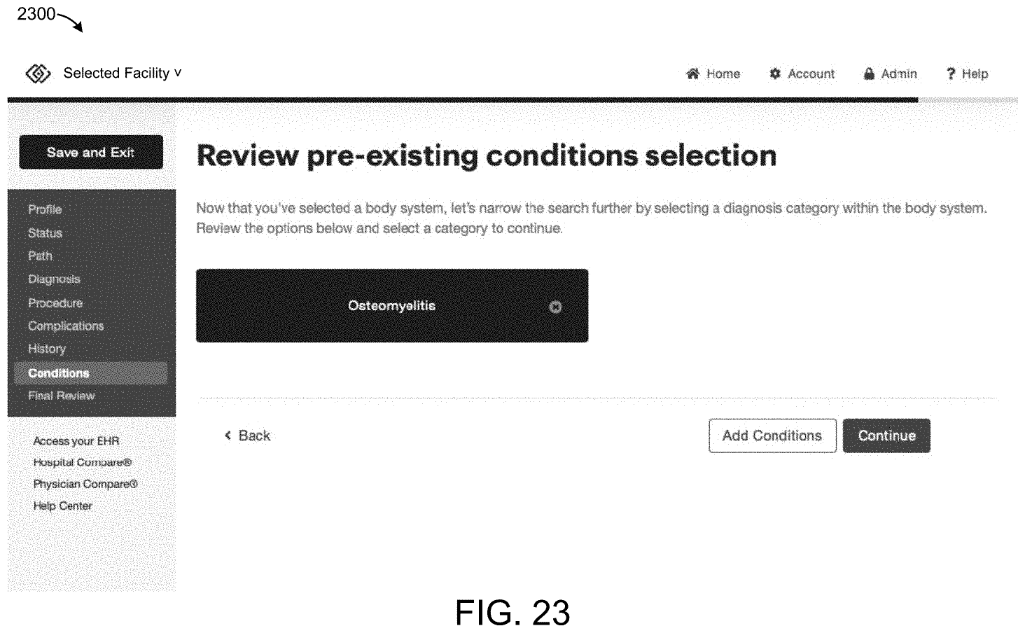

FIG. 22 is a screenshot diagram of a user interface that is responsive to the selection made at screen 2100. In an embodiment, the user selected the selectable object labeled “skin, epidermis—integumentary,” which may cause screen 2200 to appear. Screen 2200 may include one or more selectable objects, each associated with one or more types of integumentary conditions. In an embodiment, the user may select the selectable object labeled “osteomyelitis.” In response to the selection of osteomyelitis, screen 2300 shown in FIG. 23 may be displayed. In an embodiment, the selectable options at screen 2200 include types of integumentary conditions, and the selectable options displayed on screen 2300 may be specific conditions within the selected type. For example, the user may select the selectable object associated with the specific condition of osteomyelitis at screen 2300.

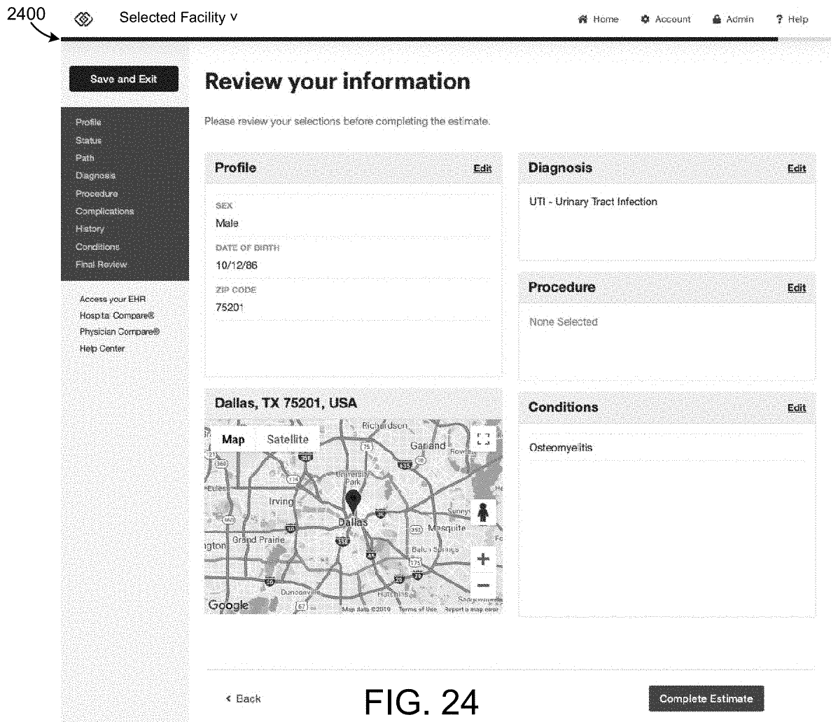

In response, screen 2400 may be presented to the user, which includes a summary of entered information for review. An embodiment of screen 2400 is illustrated in FIG. 24. Although the embodiments described above included selection of specific inputs, including specific diagnoses, specific procedures, and specific pre-existing conditions, one of ordinary skill will recognize that a wide variety of selection may be made by a user. Indeed, the selected diagnoses, conditions, and procedures may vary as widely as the ICD-10 codes for which DRGs have been defined in the system.

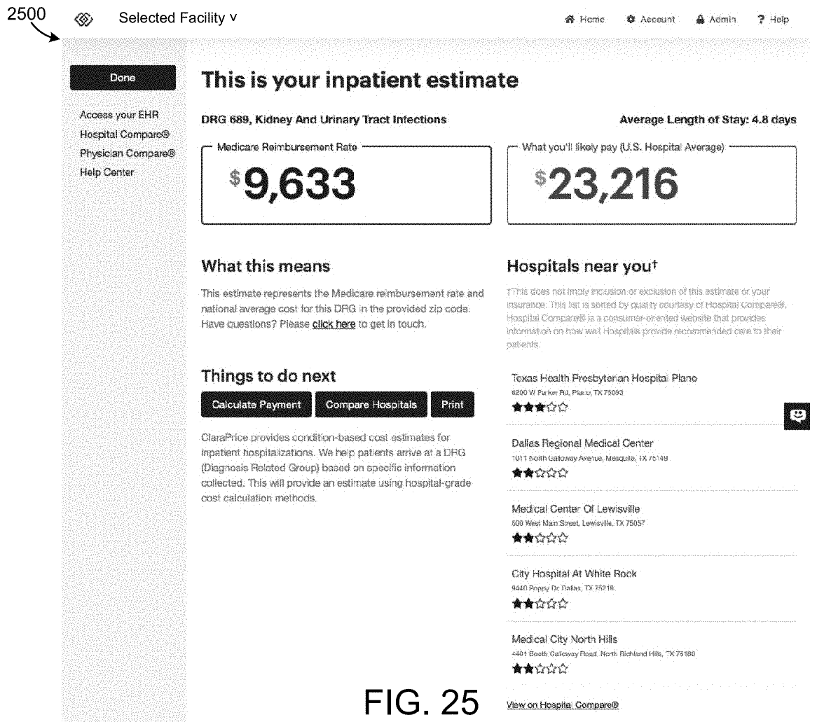

FIG. 25 is a screenshot diagram of one embodiment of a user interface for displaying health care cost estimate information. In an embodiment, screen 2500 may include an estimate of the cost of the medical procedures that is calculated by the application web server in response to the demographic data, location data, procedure or diagnosis data, and pre-existing or chronic condition data entered by the user in screens 1200-2400. In an embodiment, the price estimate may include an estimate of the total cost for the procedure, which is $23,216 in this embodiment. Also, the application server may take into account medical reimbursements based on a specified health insurance plan for the patient. In such an embodiment, the reimbursement rate may be shown. In a further embodiment, one or more suggestions for competing hospitals, clinics, other facilities, or other providers may be offered to help the user reduce cost for the procedure.

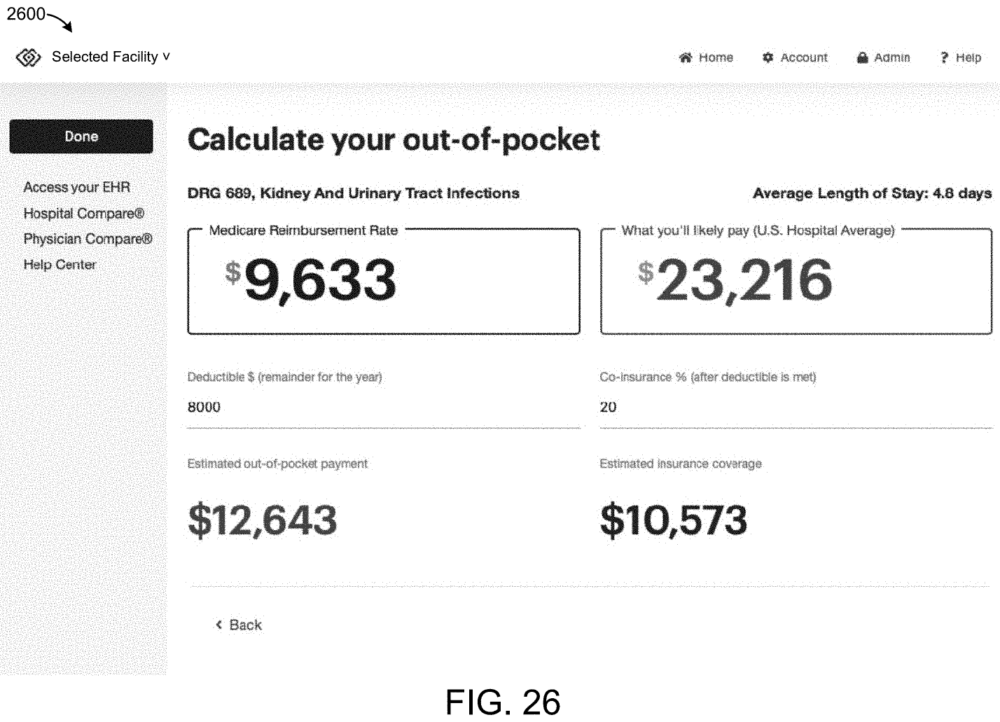

FIG. 26 is a screenshot diagram of one embodiment of an out-of-pocket cost calculator, as shown in screen 2600. Screen 2600 may prompt the user for insurance information including, e.g., insurance deductible and co-insurance coverage rates. The calculator may then use the estimates generated for screen 2500 to calculate the estimated insurance coverage and the estimated out-of-pocket cost. In addition, for verification purposes, the identified DRG identifier for the selected procedure is displayed. Also, the average length of stay in the hospital may also be displayed.

Although specific cost data has been displayed in response to a specific DRG and specific demographic and pre-existing data have been described. One of ordinary skill will recognize that alternative data may be used to identify a variety of other DRG identifiers, weighting factors, cost information, and the like. Such alternative embodiments may depend on alternative user inputs, alternative insurance information, and alternative pre-configured lists or database values.

Although the invention(s) is/are described herein with reference to specific embodiments, various modifications and changes can be made without departing from the scope of the present invention(s), as set forth in the claims below. Accordingly, the specification and figures are to be regarded in an illustrative rather than a restrictive sense, and all such modifications are intended to be included within the scope of the present invention(s). Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements. The terms “coupled” or “operably coupled” are defined as connected, although not necessarily directly, and not necessarily mechanically. The terms “a” and “an” are defined as one or more unless stated otherwise. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, device, or apparatus that “comprises,” “has,” “includes” or “contains” one or more elements possesses those one or more elements but is not limited to possessing only those one or more elements. Similarly, a method or process that “comprises,” “has,” “includes” or “contains” one or more operations possesses those one or more operations but is not limited to possessing only those one or more operations.

1. A method for condition-based health care cost estimation, comprising:
obtaining health care cost information associated with a Diagnosis-Related Group (DRG) defined by the tenth (10th) revision of the International Classification of Disease and related health problems (ICD-10) from a database;
receiving patient profile information from a remote user via a user portal interface;
converting the patient profile information into a cost estimate based on the health care cost information using a processing device; and
communicating the cost estimate to the remote user interface device.

2. The method of claim 1, wherein converting the patient profile information into a cost estimate further comprises assigning a weighting factor to the costs defined by the DRG, the weighting factor representative of possible comorbidity conditions which may factor into the cost, the possible comorbidity conditions derived from the patient profile information.

3. The method of claim 1, wherein the patient profile information further comprises at least one of an age and a date of birth for the patient.

4. The method of claim 1, wherein the patient profile information further comprises location information.

5. The method of claim 1, wherein the patient profile information further comprises identification of a biological sex of the patient.

6. The method of claim 1, further comprising receiving medical history information associated with the patient, the medical history information including identification of a pre-existing condition or a chronic condition of the patient.

7. The method of claim 1, further comprising prompting the user for identification of a diagnosed condition.

8. The method of claim 1, further comprising prompting the user for identification of a procedure to be performed.

9. The method of claim 1, further comprising calculating an estimate of coverage of an insurance or reimbursement program.

10. The method of claim 1, further comprising performing DRG grouping, wherein the DRG grouping comprises:
converting the patient profile data into ICD-10 data for DRG grouping;
posting the converted data set over HTTP API request to perform the DRG grouping;
returning the assigned DRG to the patient pricing portal over HTTP API request; and
wherein the grouped DRG data and patient profile data are used to determine the cost estimate.

11. A system for condition-based health care cost estimation, comprising:
a network interface configured to:
obtain health care cost information associated a Diagnosis-Related Group (DRG) defined by the tenth (10th) revision of the International Classification of Disease and related health problems (ICD-10) from a database; and
receive patient profile information from a remote user via a user portal interface; and
a processing device configured to convert the patient profile information into a cost estimate based on health care cost information using a processing device;
wherein the network interface is further configured to communicate the cost estimate to a remote user interface device.

12. The system of claim 11, wherein converting the patient profile information into a cost estimate further comprises assigning a weighting factor to the costs defined by the DRG, the weighting factor representative of possible comorbidity conditions which may factor into the cost, the possible comorbidity conditions derived from the patient profile information.

13. The system of claim 11, wherein the patient profile information further comprises at least one of an age and a date of birth for the patient.

14. The system of claim 11, wherein the patient profile information further comprises location information.

15. The system of claim 11, wherein the patient profile information further comprises identification of a gender of the patient.

16. The system of claim 11, further comprising receiving medical history information associated with the patient, the medical history information including identification of a pre-existing condition or a chronic condition of the patient.

17. The system of claim 11, further comprising prompting the user for identification of a diagnosed condition.

18. The system of claim 11, further comprising prompting the user for identification of a procedure to be performed.

19. The system of claim 11, further comprising calculating an estimate of coverage of an insurance or reimbursement program.

20. The system of claim 11, further comprising a DRG grouper unit configured to:
convert the patient profile data into ICD-10 data for DRG grouping;
post the converted data set over HTTP API request to perform the DRG grouping;
return the assigned DRG to the patient pricing portal over HTTP API request; and
wherein the grouped DRG data and patient profile are used to determine the cost estimate.
